SEQUENCE LISTING (14 sequences)

SEQ ID NO: 1 - DNA, 30 bp, Artificial Sequence
  Ag85B Foward primer
acaggatccgcgatagatccataccgccat

SEQ ID NO: 2 - DNA, 33 bp, Artificial Sequence
  Ag85B Reverse primer
tatgagctcgccggcgcctaacgaactctgcag

SEQ ID NO: 3 - DNA, 31 bp, Artificial Sequence
  CFP-10 Forward primer
aatgagctcatggcagagatgaagaccgatg

SEQ ID NO: 4 - DNA, 33 bp, Artificial Sequence
  CFP-10 Reverse primer
actaaagctttcagaagcccatttgcgaggaca

SEQ ID NO: 5 - DNA, 34 bp, Artificial Sequence
  hIL-12 Forward primer
ttcttatcgatatgggtcaccagcagttggtcat

SEQ ID NO: 6 - DNA, 34 bp, Artificial Sequence
  hIL-12 Reverse primer
tttttatcgatggaagcattcagatagctcatca

SEQ ID NO: 7 - protein, 15 aa, Artificial Sequence
  Ag85B241-255aa
QDAYNAAGGHNAVFN

SEQ ID NO: 8 - protein, 20 aa, Artificial Sequence
  Ag85B261-280aa
THSWEYWGAQLNAMKGDLQS

SEQ ID NO: 9 - protein, 15 aa, Artificial Sequence
  CFP-1011-25
LAQEAGNFERISGDL

SEQ ID NO: 10 - protein, 17 aa, Artificial Sequence
  Ag85B1-19aa
FSRPGLPVEYLQVPSMG

SEQ ID NO: 11 - protein, 8 aa, Artificial Sequence
  CFP-1032-39
VESTAGSL

SEQ ID NO: 12 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 13 - protein, 100 aa, Mycobacterium tuberculosis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 14 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

(19) United States
(12) Patent Application Publication
(10) Pub. No.: US 2013/0115240 A1
(43) Pub. Date: May 9, 2013

(54) RECOMBINANT BCG STRAINS WITH ENHANCED ABILITY TO INHIBIT INTRACELLULAR MYCOBACTERIAL GROWTH

(71) Applicant: National Health Research Institutes, Miaoli County (TW)

(72) Inventor: Horng-Yunn DOU, Miaoli County (TW)

(73) Assignee: National Health Research Institutes, Miaoli County (TW)

(21) Appl. No.: 13/672,013

(22) Filed: Nov. 8, 2012

Related U.S. Application Data
(60) Provisional application No. 61/557,524, filed on Nov. 9, 2011.

(51) Int. Cl. A61K 39/04

(52) U.S. Cl. CPC A61K 39/04
USPC 424/192.1; 435/252.3

(57) ABSTRACT

A recombinant bacterial cell strain is disclosed. It comprises: a) a first vector comprising a fusion transgene encoding Ag85B-CFP10 fusion protein, the fusion transgene being operably linked to a promoter effective for expression of the Ag85B-CFP10 fusion protein; and b) a second vector comprising a transgene encoding interleukin-12 (IL-12), the transgene being operably linked to a promoter effective for expression of the IL-12 protein. A method of inhibiting intracellular growth of Mycobacterium in a subject is also disclosed.

### REFERENCE TO RELATED APPLICATION

The present application claims the priority to U.S. Provisional Application Ser. No. 61/557,524 filed Nov. 9, 2011, which is herein incorporated by reference in its entirety.

### FIELD OF THE INVENTION

The present invention relates generally to vaccines, and more specifically to vaccines against mycobacterium.

### BACKGROUND OF THE INVENTION

Tuberculosis continues to be a major cause of disease and death throughout the developing world. The situation is worsened by the emergence of multidrug-resistant strains of M. tuberculosis and the MTB-HIV co-infection. Currently the present available vaccine against human TB is Mycobacterium bovis Bacille Calmette Guérin (BCG). This live attenuated vaccine has been in used for more than 80 years. However, the efficacy of BCG is variable (ranging from 0-80%).

To date, no successful vaccines have been developed which confer immunity to infection by M. tuberculosis and at the same time treat or prevent the development of symptoms of TB after exposure to M. tuberculosis, or as a result of reactivation of latent infection.

Therefore, a heretofore unaddressed need exists in the art to address the aforementioned deficiencies and inadequacies related to vaccines against tuberculosis, especially in connection with a long-lasting high protection rate.

### SUMMARY OF THE INVENTION

In one aspect, the invention relates to a recombinant bacterial cell comprising: a) a first vector comprising a first transgene encoding Ag85B and CFP10 proteins; and b) a second vector comprising a second transgene encoding interleukin-12 (IL-12).

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, tire same reference numbers are used throughout the drawings to refer to the same or like elements of an embodiment.

### BRIEF DESCRIPTION OF THE DRAWINGS

FIG. 1 shows construction of recombinant BCG expressing Ag85B/CFP-10 fusion antigen and hIL-12. Expression of the protein is driven by BCG hsp60 promoter and the arrow indicates the direction of transcription. (A) The gene fusion encoding Ag85B and CFP-10 were amplified from M. tuberculosis H37Rv chromosomal DNA. The PCR products was digested by enzymes and cloned into the E. Coli-Mycobacterium shuttle plasmid pMV261. (B) Coding sequences for hIL-12 were amplified from human IL-12 cDNA using specific primer, and cloned into E. Coli-Mycobacterium shuttle plasmid pVV16.

FIG. 2 shows Western blot analysis of recombinant BCG expressing Ag85B/CFP-10 fusion antigen. Cell lysates were analyzed for expression of recombinant antigens by immunoblotting using antibodies recognizing Ag85B (A) and CFP-10 (B). (C) Gel stained with SIMPLYBLUE™ SAFE STAIN dye. rBCG-1 is pMV261-LAg85B/CFP10 rBCG, rBCG-2 is pMV261-LAg85B/CFP10+pVV16-hIL-12rBCG.

FIG. 3 shows ELISA analysis of recombinant BCG expressing hIL-12.1 and 2 represent results from two independent clones transformed with pMV261-LAg85B/CFP10+pVV16-hIL-12 rBCG. *p-value<0.05.

FIG. 4 shows growth curves of BCG and rBCG. pMV261-LAg85B/CFP10 rBCG, rBCG-2 is pMV261-LAg85B/CFP10+pVV16-hIL-12 rBCG.

FIG. 5 shows functional characterization of peptide-specific CD4+ and CD8+ T-cell responses in the spleen of vaccinated mice analyzed by intracellular cytokine staining. CD4+ and CD8+ T-cell functional status in C57BL/6 mice vaccinated by BCG or recombinant BCG was determined by using a three-function assay that measured expression of IFN-γ, TNF-α and IL-2 following stimulation with antigenic peptides. (A) Relative proportions of CD4+ T cells that secrete different amounts of cytokines. (B) Relative proportions of CD8+ T cells that secrete different amounts of cytokines. rBCG-1 is pMV261-LAg85B/CFP10 rBCG; rBCG-2 is pMV261-LAg85B/CFP10+pVV16-hIL-12 rBCG. 4W==4 weeks, 8W==8 weeks.

FIG. 6 shows peptide-specific CD4+ and CD8+ T-cell responses in the lungs of vaccinated mice analyzed by intracellular cytokine staining. (A) Number of CD4+ T cells in the lungs of C57BL/6 mice showing intracellular expression of IFN-γ at weeks 4, 8 and 12 after vaccination. (B) Number of IFN-γ+ CD8+ T cells in the kings of C57BL/6 mice at weeks 4, 8 and 12 after vaccination. , vaccination by PBS; ▪, vaccination by BCG; ▴, vaccination by pMV261-LAg85B/CFP10 rBCG; and ♦, vaccination by pMV261-LAg85B/CFP10+pVV16-hIL-12 rBCG.

FIG. 7 shows peptide-specific CD4+ combine CD44+ and CD8+ combine CD44+ T-cell responses in the lung and spleen of vaccinated mice analyzed by intracellular cytokine staining. (A) Numbers of CD4+ CD44+ T cells expressing intracellular IFN-γ in the lungs of C57BL/6 mice at week 6 after vaccination. (B) Number of IFN-γ+ CD8+ CD44+ T cells in the kings of C57BL/6 mice at week 6 after vaccination. (C) Numbers of CD4+ CD44+ T cells expressing intracellular IFN-γ in the spleen of C57BL/6 mice at week 6 after vaccination. (D) Numbers of IFN-γ+ CD8+ CD44+ T cells in the spleen from C57BL/6 mice at week 6 after vaccination, * * the number of dots was significantly higher than for the group vaccinated with BCG (* represents p-value<0.05) rBCG-1, refers to vaccination by pMV261-LAg85B/CFP10 rBCG; rBCG-2 refers to vaccination by pMV261-LAg85B/CFP10+ pVV16-hIL-12 rBCG.

FIG. 8 shows antibody response against Ag85B in mice immunized with PBS, BCG and rBCG. C57BL/6 mice were immunized with PBS, BCG, rBCG-1 or rBCG-2, and sacrificed after 4, 8 or 12 weeks to prepare serum for examining the ratio of specific IgG2b/IgG1, rBCG-1 and rBCG-2 refer to pMV261-LAg85B/CFP10 rBCG and pMV261-LAg85B/CFP10+ pVV16-hIL-12 rBCG, respectively.

FIG. 9 shows splenocytes recovered from immunized mice. Murine bone marrow macrophages that were infected with M. tuberculosis were cocultured with splenocytes taken from mice immunized with BCG or the rBCG and the growth, of M. tuberculosis was monitored over an 11-day culture period. Data represents ±SEM of mice. Where * represents p-value<0.05.

FIG. 10 shows the analysis of antigen-specific IFN-γ production. The immune response was measured by an ELISPOT assay. Cells from Spleen (A,C and E) and lung (B, D and F) were isolated from mice immunized with PBS, BCG, rBCG 1 or rBCG2 and stimulated with PBS (A,B), 10 μg/ml PPD(C, D) and 5 μg/ml Ag85b and 5 μg/ml CPF10 (E, F). Number of cells secreting IFN-γ in the suspension of single cell was counted *, the number of dots were significantly higher than those group immunized with BCG and rBCG1 strains (P<0.05).

FIG. 11 shows antibody response against Ag85b and CFP10 in mice immunized with PBS, BCG, rBCG1 or rBCG2. Four groups of mice were immunized with PBS, BCG, rBCG1 and rBCG2 respectively, and sacrificed after 4, 8 and 12 weeks to prepare serum for examining the antibody response of Ig (A,B) and the ratio of IgG2a/IgG1 (C,D).

FIG. 12 shows vaccine induced inhibition of Mycobacterium tuberculosis H37Rv growth seen on day 0, 4 and 7 co-culture of splenocytes and M. tuberculosis H3Rv-infected macrophages. After 4 (A) or 8 weeks (B), four groups of mice vaccinated with PBS, BCG, rBCG1 and rBCG2 were scarified. The spleen was aseptically removed, M. tuberculosis H37Rv infected macrophages were co-cultured with splenocytes from mice immunized with vaccine. At specified time, the bacteria uptake by murine bone marrow-derived macrophages was determined.

### DETAILED DESCRIPTION OF THE INVENTION

### Definitions

The terms used in this specification generally have their ordinary meanings in the art, within the context of the invention, and in the specific context where each terra is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a terra is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

Bacillus Calmette-Guérin (or Bacille Calmette-Guérin, Mycobacterium bovis Bacillus Calmette-Guérin, BCG) is a vaccine against tuberculosis that is prepared from a strain of the attenuated (weakened) live bovine tuberculosis bacillus, Mycobacterium bovis, that has lost its virulence in humans by being specially subcultured (230 passages) in an artificial medium for 13 years, and also prepared from Mycobacterium tuberculosis. The bacilli have retained enough strong antigenicity to become a somewhat effective vaccine for the prevention of human tuberculosis. At best, the BCG vaccine is 80% effective in preventing tuberculosis for a duration of 15 years.

Antigen 85B Mycobacterium tuberculosis is the most abundant protein secreted by M. Tuberculosis, as well as a potent immunoprotective antigen and a leading drag target. Ag85 induces strong T-cell proliferation and IFN-γ secretion in most healthy individuals exposed to M. tuberculosis, in BCG-vaccinated mice and humans whereas the antibody against Ag85 are more prevalent in active tuberculosis patients with decreased cellular immune response.

Culture filtrate protein-10 (CFP-10) is also known as ESAT-6-like protein esxB or secreted antigenic protein MTSA-10. 10 kDa culture filtrate antigen CFP-10 is a protein that is encoded by the esxB gene. It is a 10 kDa secreted antigen, from Mycobacterium tuberculosis. It forms a 1:1 heterodimeric complex with ESAT-6. Both genes are expressed from the RD1 region of the bacterial genome and play a key role in the virulence of the infection.

As used herein, the term “overexpressing” or “increased expression” shall generally means the expression of a specific protein in a subject vaccinated with a rBCG according to the invention is higher than that in a subject vaccinated with BCG or PBS.

The term “vector” is exchangeable with “plasmid”, which is used to transfer genetic material to a target cell.

The invention relates to a discovery that mice vaccinated with rBCG expressing Ag85B/CFP10 fusion protein together with the immune stimulatory cytokine interleukin (IL)-12 exhibited a robust immune response that was qualitatively superior to that elicited by licensed BCG vaccine. Furthermore, it was discovered that large numbers of specific Th1 cells were present in the lung and spleen of the rBCG-vaccinated mice.

Mycobacterium tuberculosis continues to be a leading cause of human deaths due to an infectious agent. Current efforts are focused on making better TB vaccines. The protective immunity against M. tuberculosis is mediated mainly by Th1-type CD4+ T cells and CD8+ T cells. These T cells secrete large amounts of cytokines, including gamma interferon (IFN-γ) and tumor necrosis factor alpha (TNF-α), resulting in enhanced macrophage bactericidal activity and prevention of bacterial dissemination to the bloodstream and other tissues.

Development of recombinant BCG (rBCG)-based vaccines over-expressing proven immuno-dominant antigens of M. tuberculosis are a promising approach to improvement of the performance of BCG, recombinant BCG strains can be engineered to express immunodominant or specific antigens, such as Ag 85B, AG85 complex, a family of 30-32 kDa proteins (Ag85A, Ag85B and Ag85C), early secreted antigenic target-6 kDa (ESAT-6).

We describe the generation and immunological characterization of recombinant BCG (rBCG). This rBCG was generated by incorporating an expression plasmid encoding two mycobacterial antigens (Ag85B and CFP10) and human interleukin (IL) IL-12 into a BCG strain. Immunogenicity studies in mice showed that recombinant BCG coexpressing Ag85B, CFP10 and interleukin-12 induces a robust immune response in mice (rBCG::Ag85B-CFP10-IL12) could induce higher specific antibody titers and significantly increase the cellular immune response than either BCG or rBCG-Ag85B-CFP10. The rBCG vaccine promotes a T cell response against MTB that is characterized by a high proportion of polyfunctional and memory T cells in spleen and lung. Our results showed that the better immunogenicity and mycobacterial growth inhibition of rBCG::Ag85B-CFP 10 plus IL-12 than that of BCG may make the rBCG the preferred vaccine candidate against TB.

In one aspect, the invention relates to a recombinant bacterial cell strain, which comprises: a) a first vector comprising a fusion transgene encoding Ag85B-CFP10 fusion protein, the fusion transgene being operably linked to a promoter effective for expression of the Ag85B-CFP10 fusion protein; and b) a second vector comprising a transgene encoding interleukin-12 (IL-12), the transgene being operably linked to a promoter effective for expression of the IL-12 protein.

In another aspect, the invention relates to a recombinant Bacille Calmette-Guerin (BCG) vaccine strain, which comprises: a) a fusion transgene encoding Ag85B-CFP10 fusion protein, the fusion transgene being operably linked to a promoter effective for an increased expression of Ag85B and CFP10 proteins; and b) a transgene encoding IL-12 protein, the transgene being operably linked to a promoter effective for expression of the IL-12 protein.

Further in another aspect, the invention relates to a method of inhibiting intracellular growth of Mycobacterium in a subject. The method comprises administering to the subject the aforementioned recombinant BCG vaccine strain or bacterial cell strain in an amount effective for inhibition of Mycobacterium growth in the lung cells of the subject. The recombinant BCG causes an increased expression of interferon-γ and/or an increased number of CD4 and CD8 T cells in the spleen and/or lung tissue of the subject, in one embodiment of the invention, the subject has Mycobacterium tuberculosis infection.

Yet in another aspect, the invention relates to a recombinant Bacille Calmette-Guerin (BCG) vaccine strain, which expresses and secretes human IL-12 protein, and exhibits an increased expression of Ag85B and CFP10 proteins as compared to a BCG vaccine strain.

In one embodiment of the invention, the aforementioned recombinant bacterial cell strain expresses or overexpresses the Ag85B-CFP10 fusion protein and the IL-12 protein.

The aforementioned recombinant bacterial cell strain may be a recombinant Mycobacterium cell. The recombinant bacterial cell strain may be a recombinant Mycobacterium tuberculosis cell. Alternatively, the recombinant bacterial cell strain may be a recombinant Mycobacterium bovis cell. The recombinant bacterial cell strain may be a recombinant Bacille Calmette-Guerin (BCG).

In another embodiment of the invention, the aforementioned fusion transgene is under the control of a heat shock protein (HSP) promoter. The HSP promoter may be a Mycobacterium bovis HSP60 promoter.

In another embodiment of the invention, the recombinant BCG vaccine strain expresses the IL-12 protein in a non-fusion protein form. The recombinant BCG vaccine strain has at least, one of the following characteristics: a) eliciting interferon γ-producing T cells and IgG2a antibody against Ag85b or CFP10; and b) conferring stronger protective efficacy against M. tuberculosis H37Rv infection compared with BCG vaccine.

In another embodiment, of the Invention, the recombinant BCG vaccine strain expresses human IL-12 protein.

In another embodiment, the protection efficacy of the rBCG vaccine according to the invention was 6 times that of the conventional BCG.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Methods and Materials

### Bacterial Strains and Cultures

Mycobacterium bovis BCG (Tokyo 172) and recombinant BCG strains (rBCG1 and rBCG2) and M. tuberculosis H37Rv were grown on Middlebrook 7H9 medium (Difco laboratories, Detroit, Mich., USA) supplemented with 0.5% glycerol, 0.05% TWEEN® 80, and 10% albumin-dextrose-catalase (ADC) or on solid 7H11 medium (Difco laboratories) supplemented with oleic-acid-albumin-dextrose-catalase (OADC). OADC enrichment was added to 7H10 and 7H11 basic medium to enhance the growth of mycobacteria. When required, the antibiotic kanamycin (Km) was added at a concentration of 25 μg/ml. Escherichia coli DH5a was grown in Luria-Bertani medium and used for cloning.

### Mice and Immunization

The female C57BL/6 and C3H/HeJ mice (aged 6-8 weeks) used in this study were purchased from National Laboratory Animal Center in Taiwan. All the animals were kept under specific-pathogen-free conditions. The mice (five per group) were immunized subcutaneously with 1×10⁷ CFU of BCG or rBCG strains in 100 μl phosphate buffered saline (PBS). A control group received 100 μl PBS. The mice were sacrificed to prepare sera and splenocytes. All animal works were approved by institute animal experimentation committee and performed in accordance with the guidelines of the institute committee.

Mice were sacrificed to conduct the analysis of immune responses at 4, 8, and 12 weeks after immunization.

Construction of rBCG

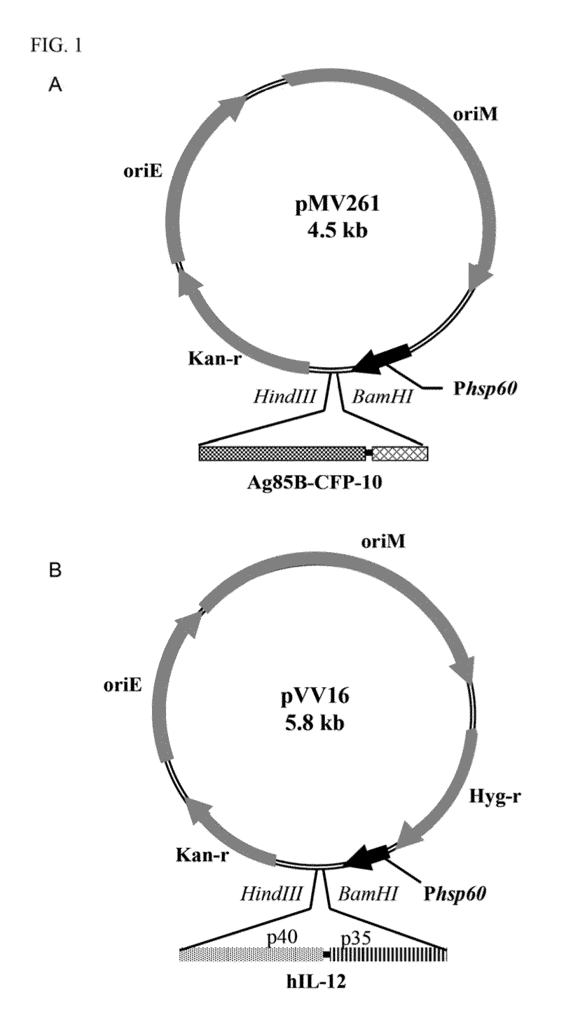

Coding sequences for Ag85B (SEQ ID NO: 12), CFP-10 (SEQ ID NO: 13) and human IL-12 (SEQ ID NO: 14) were amplified from M. tuberculosis H37Rv genomic DNA and human IL-12 open reading frame (pORF-hIL-12, INVIVOGEN™), respectively, by PCR using the primers and annealing temperatures shown in Table 1. The ag85b and cfp-10 coding regions were linked and inserted into the Bam HI and Hind III sites of the mycobacterial-E. coli shuttle vector pMV261 (FIGS. 1A-B). This vector permitted the Ag85b-CFP-10 fusion expression under the M. bovis HSP60 promoter. The IL-12 coding regions were cloned into the low copy number mycobacterial-E. coli shuttle vector pVV16 (FIG. 1B). All inserted genes were confirmed by sequencing. The two recombinant plasmids pMV261-LAg85B/CFP10 and pVV16-hIL-32 were used to transform BCG by electroporation. The transformed BCG cells were plated on 7H11 medium supplemented with 30 μg/mL kanamycin or 100 μg/mL hygromycin, then grown at 37° C. for 3 weeks. Individual colonies were picked and grown in Sauton medium (0.25 g MgSO4-7H2O, 0.25 g K2HPO4, 1 g citric acid, 4 g sodium glutamate, 30 mL glycerol, 5 mg ZnSO4 and 25 mg ferrum-ammonium citrate in 500 mL) containing 30 μg/mL kanamycin or 100 μg/mL hygromycin. After 2 weeks' growth, protein expression was induced by heating at 45° C. for 60 min. The bacterial cells were centrifuged at 8000 g for 20 min. Twelve micrograms of the concentrated culture supernatants were analyzed for expression of recombinant antigens by immunoblotting using anti-Ag85B and anti-CFP10 polyclonal antibody (DIATHEVA). The recombinant vaccines were grown in parallel in 100 mL Middlebrook 7H9 medium supplemented with 0.5% glycerol, 0.05% Tween 80, 10% ADC and 30 μg/mL kanamycin or 100 μg/mL hygromycin. Bacteria were collected by centrifugation at 8000 g for 20 min and washed once with 50 mM phosphate-buffered saline (PBS) (pH 7.0) before resuspension in 2 mL of PBS supplemented with 25% glycerol. The bacterial suspensions were divided into aliquots and frozen at −80° C. for later use. A single aliquot was defrosted for quantification of each vaccine lot. Table 1 lists primers and PCR conditions used to engineer the recombinant BCG (LIN, C.-W., SU, I.-J., CHANG, J.-R., CHEN, Y.-Y., LU, J.-J. and DOU, H.-Y. “Recombinant BCG coexpressing Ag85B, CFP10, and interleukin-12 induces multifunctional Th1 and memory T cells in mice” APMIS. 2012 January; 120(1):72-82, which is incorporated herein by reference in its entirety).

TABLE 1

| Target | Gene | | | Annealing |
|---|---|---|---|---|
| gene | size | Forward primer | Reverse primer | temperature |
| Ag85B | 1153 | acaggatccgcgatagatccataccgccat | tatgagctcgccggcgcctaacgaactctgc | 58° C. |
| | bp | (SEQ ID NO: 1) | ag (SEQ ID NO: 2) | |
| CFP-10 | 303 | aatgagctcatggcagagatgaagaccgatg | actaaagctttcagaagcccatttgcgaggac | 62° C. |
| | bp | (SEQ ID NO: 3) | a (SEQ ID NO: 4) | |
| hIL-12 | 1611 | ttcttatcgatatgggtcaccagcagttggtcat | tttttatcgatggaagcattcagatagctcatca | 58° C. |
| | bp | (SEQ ID NO: 5) | (SEQ ID NO: 6) | |

### ELISA

ELISA plates were coated overnight at 4° C. with 5 μg/mL Ag85B recombinant protein (DIATHEVA). The plates were blocked with 250 μL/well PBS containing 1% bovine serum albumin for 1 h at room temperature and washed with PBS containing 0.05% TWEEN® 20 three times. Sera were added at serial twofold dilutions (beginning at a 1/500 dilution) for 2 h at room temperature and washed, followed by addition of 100 μL/well horseradish peroxidase-conjugated rabbit anti-mouse IgG (Jackson Immuno Research), IgG1 and IgG2b diluted at 1/10 000, 1/1000 and 1/1000 in PBS, respectively. Plates were incubated for 1 h at room temperature, washed and developed with TMB substrate solution (Immunology Consultants Laboratory, Inc). Antibody titers are expressed as reciprocal end-point titers. Reactions were stopped by addition of 50 μL/well of 0.3 M H₂SO₄ and were read on an ELISA plate reader at 450 nm. IL-12 secreted by rBCG was measured by using a high-sensitivity IL-12p70 ELISA kit (DIACLONE) according to the manufacturer's instructions.

### Cell Isolation and Peptides

Splenocytes and lung mononuclear cells (MNCs) were cultured in RPMI-1640 medium supplemented with 10% heat-inactivated fetal bovine serum (FBS), L-glutamine, 2-mercaptoethanol, penicillin and streptomycin. Lung tissue was minced and incubated with stirring for 30 min at 37° C. in HBSS with 1.3 mmol/L EDTA, followed by treatment for 1.5 h at 37° C. with 150 U/mL collagenase type 1 (Invitrogen Life Technologies) in RPMI-1640 with 10% FBS. The resulting suspension was pelleted by centrifugation, resuspended in 45% Percoll (Pharmacia), layered on 66.6% Percoll, and centrifuged at 600 g. Cells at the gradient interface were harvested and washed extensively before use. The splenocytes and lung MNCs were stimulated with 20 μg/mL of tuberculin-purified protein derivative (PPD) (SSI, Copenhagen, Denmark). Each peptide was at a final concentration of 8 μg/mL during the stimulation. In some experiments cells were stimulated with: the CD4 peptides Ag85B₂₄₁₋₂₅₅ₐ QDAYNAAGGHNAVFN (SEQ ID NO: 7), Ag85B₂₆₁₋₂₈₀ₐₐ THSWEYWGAQLNAMKGDLQS (SEQ ID NO: 8) for C57BL/6 mice (H-2b), and CFP-10₁₁₋₂₅LAQEAGNFERISGDL (SEQ ID NO: 9) for C3H mice (H-2K); and the CD8 peptides Ag85B₁₋₉₉ₐₐFSRPGLPVEYLQVPSMG (SEQ ID NO: 10) for C57BL/6 mice (H-2b) and CFP-10₃₂₋₃₉VESTAGSL (SEQ ID NO: 11) for C3H mice (H-2K), all at 5 μg/ml. These peptides contain the dominant epitopes of antigen 85B and CFP-10. Peptides were synthesized by ANGENE®, Inc.

### Intracellular Cytokine Staining

Splenocytes and lung MNCs were stimulated with PPD or pooled 85B peptides for 1 h at 37° C. Golgi Plug (eBioscience) was added according to the manufacturer's instruction and cells were incubated for an additional 4 h before intracellular cytokine stamina. Oils were washed and stained for CD4/CD8/CD44 and IFN-γ, IL-2 and TNF-α (eBioscience) using the BD Cytofix/Cytoperm kit according to the manufacturer's Instructions. Treatment with Cytoperm/wash (BD Biosciences) was applied twice before staining with monoclonal antibody mixtures (mAb). These Samples were fixed with PBS 1% paraformaldehyde, run on a FACSalibur flow cytometer, and data were analyzed with CellQuest software (BD Biosciences). Three individual mice were analyzed per group. Cytokine frequency and number presented were after background subtraction of an identically gated population of cells from the same sample, incubated with medium only.

### Growth Inhibition Assay

The assays were performed by co-culturing splenocytes from vaccinated C57BL/6 mice with M. tuberculosis-infected macrophages. For the assay, murine bone marrow macrophages (BMMφ) were the target cells for M. tuberculosis H379Rv infection. BMMφ were prepared by flushing the femurs of C57BL/6 female mice with Dulbecco's modified Eagle's medium (DMEM) (INVITROGEN™, Grand Island, N.Y., USA) supplemented with 10% heat-inactivated FBS, 10% L-929a conditioned medium, 1mM 1-glutamine, 1 mM HEPES buffer, 1 mM non-essential amino acids, and 1 mM sodium pyruvate (complete DMEM (cDMEM)). BMMφ were plated at 1×10⁶ per well of a 24-well plate (COSTAR®, CORNING®, NY, USA) in cDMEM supplemented with 100 units/ml of M-CSF (ProSpec-Tany TechnoGene, Ltd, East Brunswick, N.J., USA) on day 1 and 3 and incubated at 37° C. in 5% CO₂ for 7 days. BMMφ were infected with M. tuberculosis H37Rv at a multiplicity of infection of 5:1 (bacterium to BMMφ ratio) for 4 h at 37° C. in 5% CO2. The wells were then washed three times with PBS to eliminate extracellular and non-adhering bacteria. Following the last wash, the PBS was replaced, with fresh cDMEM medium at 37° C. in 5% CO₂. Splenocytes from immunized mice with BCG, rBCG1 and rBCG2 vaccines were used to evaluate the protective ability of the rBCG vaccines against M. tuberculosis infection in BMMφ. At specific points (4, 8 and 12 weeks) after vaccination, spleens from immunized mice were aseptically removed and placed in sterile PBS buffer temporarily. As described by Parra et. al, spleen obtained from mice was passed through a 100 μm cell strainer (BD BIOSCIENCES™, Mountain View, Calif., USA) to generate a single cell suspension, and incubated in ammonium-potassium-chloride (ACK) lysis buffer (0.15M NH₄Cl, 1 mM KHCO₃) for 4 minutes. Following the cells were re-suspended in cold DMEM and added to culture flasks for 2 hours incubation at 37° C. to remove adherent cells. Non-adherent splenocytes were harvested by gently pipetting the suspensions. The viability of the cells was assessed by exclusion of trypan blue. After that, 5×10⁶ of non-adherent splenocytes were overlaid on M. tuberculosis infected BMMφ. At selected time points, the bacteria uptake by BMMφ were determined by lysing a fraction of the cells with 0.1% saponin and counted the diluted cell lysates by plating.

### IFN-γ Enzyme-Linked Immunospot (ELISPOT) Assay

Peptides Ag85B₂₄₁₋₂₅₅ₐ QDAYNAAGGHNAVFN (SEQ ID NO: 7), Ag85B₂₆₁₋₂₈₀ₐₐ THSWEYWGAQLNAMKGDLQS (SEQ ID NO: 8), Ag85B₁₋₁₉ₐₐ FSRPGLPVEYLQVPSMG (SEQ ID NO: 10), CFP10₁₁₋₂₅ₐₐ LAQEAGNFERISGDL (SEQ ID NO: 9) and CFP10₃₂₋₃₉ VESTAGSL (SEQ ID NO: 11) were used as antigen stimuli to perform IFN-γ ELISPOT assay. The C3H/HeJ mice were euthanized and their spleens and lungs were removed aseptically. Cells from spleens and lungs were diluted in culture medium containing an appropriate stimulus (10 μg/ml tuberculin purified protein derivative (PPD, Statens Serum Institute, Copenhagen, Denmark), 5 μg/ml Ag85b/CFP10) and placed in the wells of the ELISPOT plate at a density of 5×10⁵ cells/well. The mouse IFN-γ ELISPOT kit was used to determine the relative number of IFN-γ-expressing cells in the suspension of single cell following the manufacturer's instructions (C-Cytech biosciences, Utrecht, Netherlands). The spots were visualized and counted. Wells with fewer than 10 spots were not used for calculations.

### Antibody Isotype Analysis

For evaluation of the Th1 and Th2 immune response, sera from the immunized C3H/HeJ mice were collected and determined by an indirect enzyme-linked immunosorbent assay (ELISA) assay. ELISA plates were coated overnight at 4° C. with recombinant protein (Ag85b or CFP10, DIATHEVA, Fano, Italy) at a final concentration of 5 μg/ml. The coated plates were blocked with 200 μl PBS buffer containing 3% bovine serum albumin at 37° C. for 1 hour. Following the plates were washed with PBS containing 0.05% TWEEN® 20 three times. Serum was added for 2 hours incubation and washed, followed by addition of horseradish peroxidase-conjugated rabbit anti-mouse IgG, IgG1 and IgG2a antibody (Jackson ImmunoResearch Laboratories Inc, West Grove, Pa., USA), respectively. Plates were incubated at 37° C. for 1 hour, washed and developed by o-phenylenediamine and hydrogen peroxide substrate. Reactions were stopped by addition of 50 μl/well of 1 N H₂SO₄ and were measured on an ELISA plate reader at 492 nm.

### Statistical Analysis

Data are presented as mean standard error (SE) of at least three independent experiments. Students/test was used to determine if there is a significant difference between two groups. A p value less than 0.05 were considered significant.

### Results:

Stable and High Expression of Ag 85B-CFP-10-IL12 from rBCG

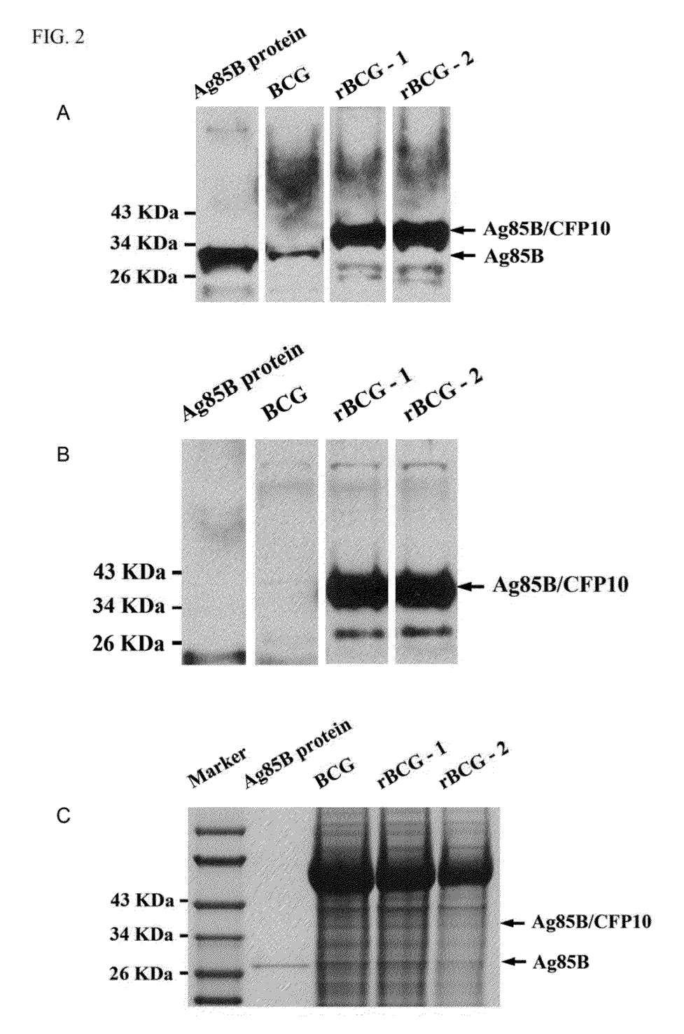

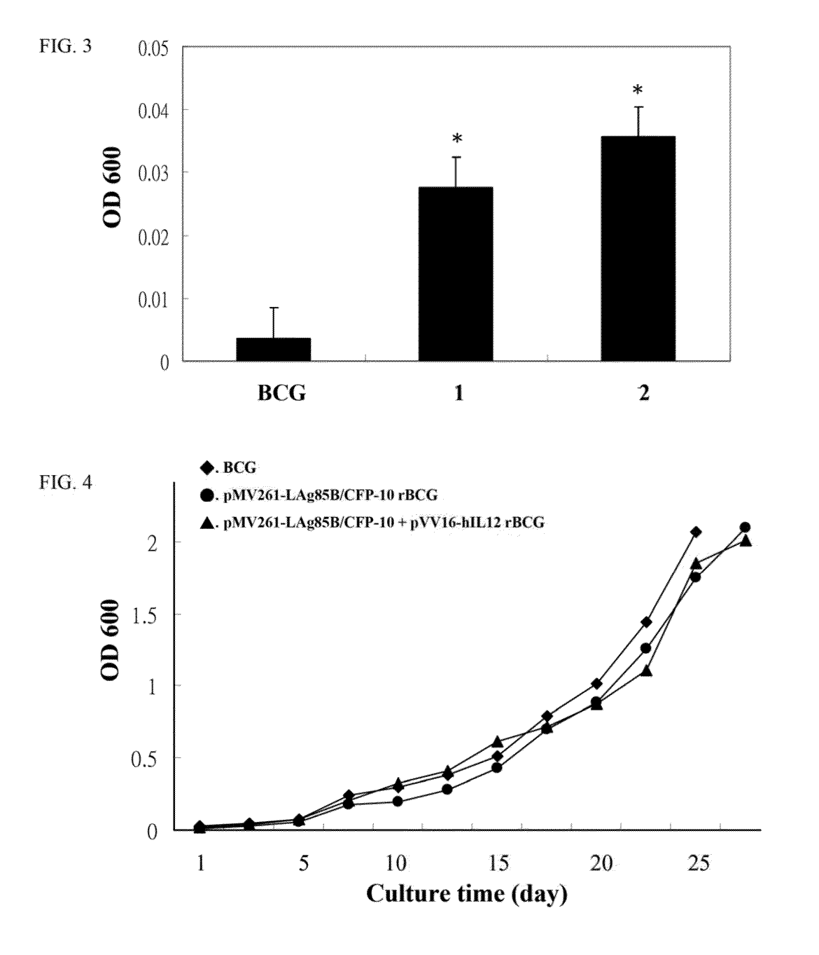

M. tuberculosis Ag85B and CFP-10 were amplified from H37Rv chromosomal DNA. The PCR product was digested by different enzymes and dotted into the E. coli-Mycobacterium shuttle vector pMV261 (conferring resistance to kanamycin), resulting in the recombinant plasmid pMV Ag85B-CFP10 (FIG. 1A). The rBCG::Ag85B-CFP10 strain was obtained by transformation of BCG with the recombinant plasmid. Immunoblot was used to analyze over expression of the Ag85B-CFP10 fusion protein (FIG. 2). The shuttle plasmid pVV16 (FIG. 1B), containing the gene encoding murine single-chain IL-12, was constructed and confirmed by DNA sequencing. pVV16-IL12 plasmid (conferring resistance to kanamycin and hygromycin) was used to transform the rBCG::Ag85B-CFP10 strain by electroporation, and cells resistant to both hygromycin and kanamycin were selected. Sandwich ELISA was used to measure the IL-12 activity of rBCG containing Ag85B-CFP10 plus IL-12 (FIG. 3). Thus, Ag85B-CFP10 fusion protein and IL-12 were confirmed to be co-expressed in rBCG::Ag85B-CFP10-IL12.

Growth of rBCG In Vitro

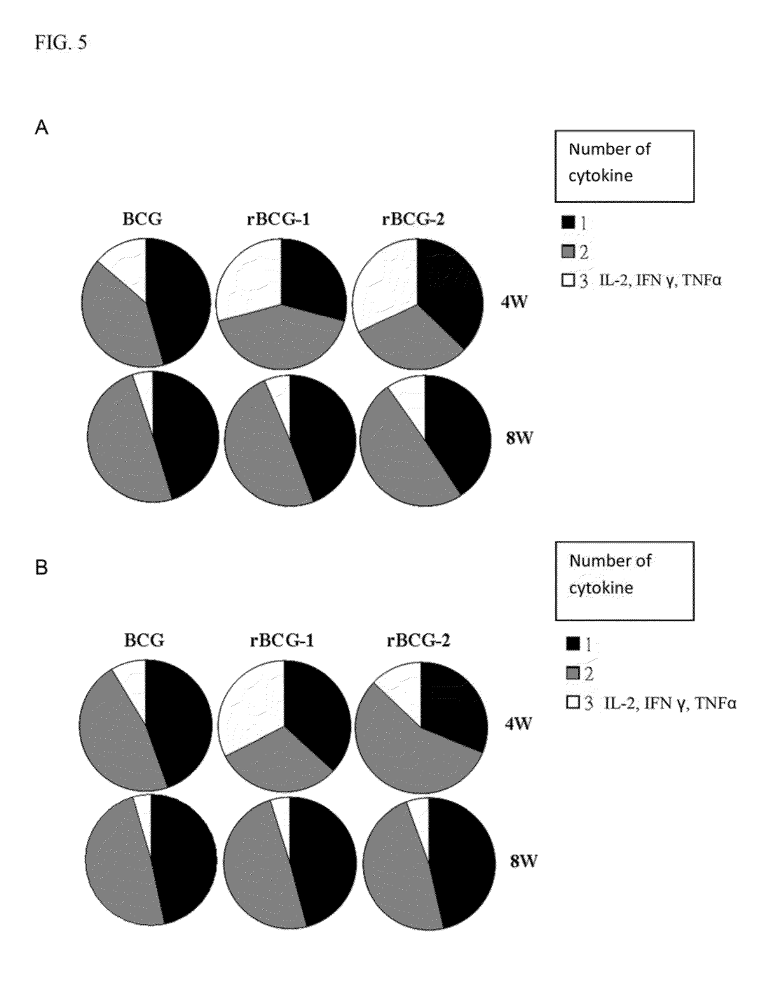

There was no significant difference in the growth curves between normal BCG and rBCG in vitro (FIG. 4).

Cytokine Production of Splenocytes from Vaccinated Mice

Six weeks after vaccination, splenocytes from BCG- and rBCG-immunized mice produced high levels of IFN-γ. The frequency of IFN-γ+ CD4 and CD8 T cells, measured by flow cytometry, showed a highly significant correlation with the frequency of cells secreting IFN-γ observed in direct ex vivo ELISPOT assays (data not shown). Virtually all IFN-γ CD4 T cells were multifunctional (FIG. 5). Responding cells were classified as 3+, 2+ or 1+ (number of cytokine) populations according to the expression profile of the functional markers IFN-γ, IL-2, and TNF-α,

T-Cell Populations in Mice after Immunization with rBCG

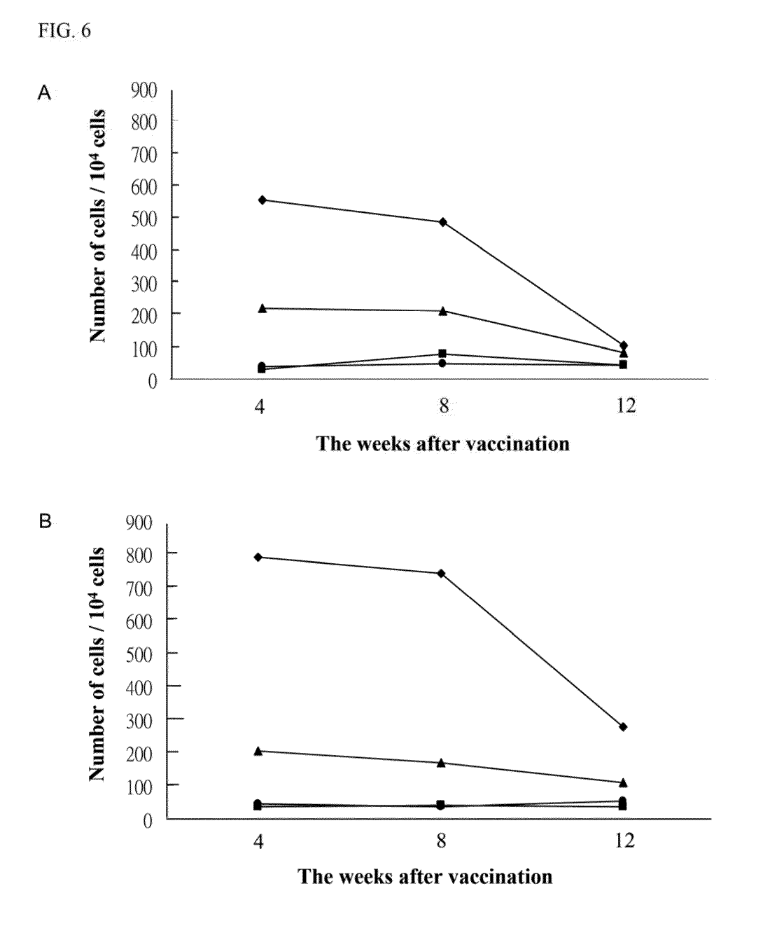

The kinetics of T-cell subpopulations in the spleen and lungs of mice after rBCG:: Ag85B-CFP10-IL12 immunization was examined. The numbers of IFN-γ-producing CD4+ and CD8+ T cells in the spleen and lungs of mice immunized with rBCGAg85B-CFP10-IL-12 were significantly higher than those of mice immunized with BCG (P<0.05) (FIG. 6). Surprisingly, the levels of IFN-γ-producing CD4+ and CD8+ T cells in the spleen and longs of mice immunized with rBCG Ag85B-CFP10-IL12 were also significantly higher than the levels in mice immunized with BCG over the course of immunization (P<0.05). These results suggested that rBCG::Ag85B CFP10-IL12 vaccination could induce greater levels of Ag-specific IFN-γ-producing CD8+ and CD4+ T-cell responses than could BCG vaccination.

### Lung Responses

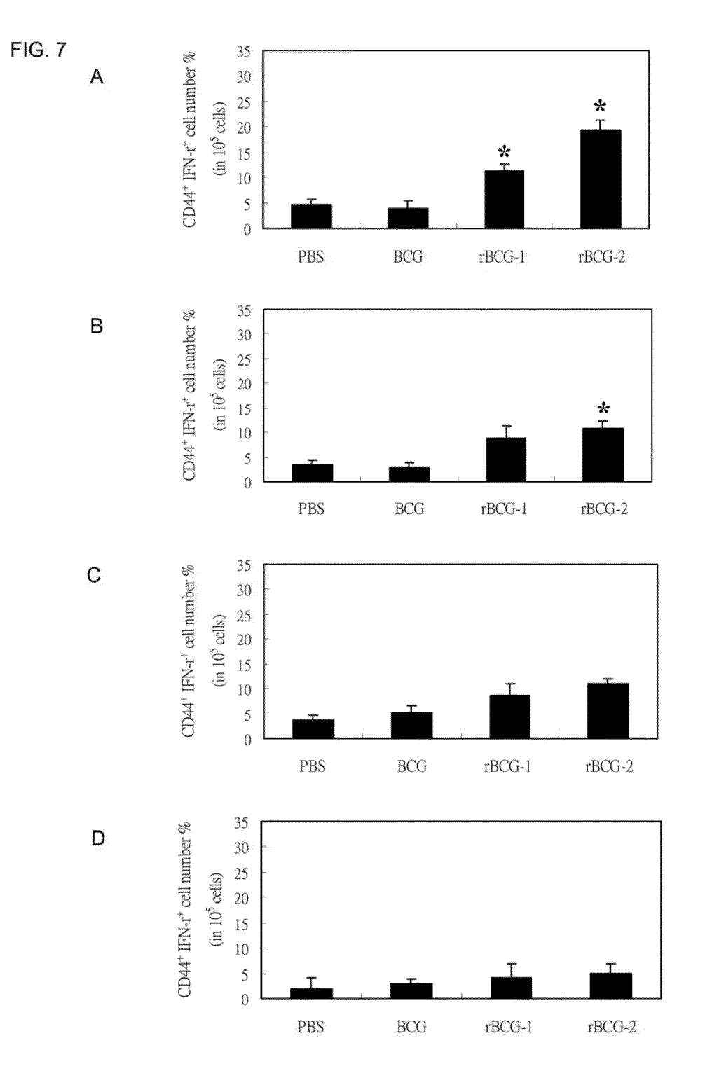

We next examined the kinetics of the T-cell subpopulations in the kings of mice after rBCG immunization. The numbers of CD4 and CD8 T cells in the lungs of mice immunized with rBCG peaked at 4 weeks (FIG. 6). The numbers of Ag85B-specific CD4+ and CD8+ T cells in the lungs were significantly greater in mice immunized with rBCG (FIG. 7). Taken together, these data suggest that rBCG::Ag85B-CFP10-IL12 vaccination enhanced not only the expansion, but also the maintenance of Ag-specific CD4+ and CD8+ T cells.

### Humoral Responses

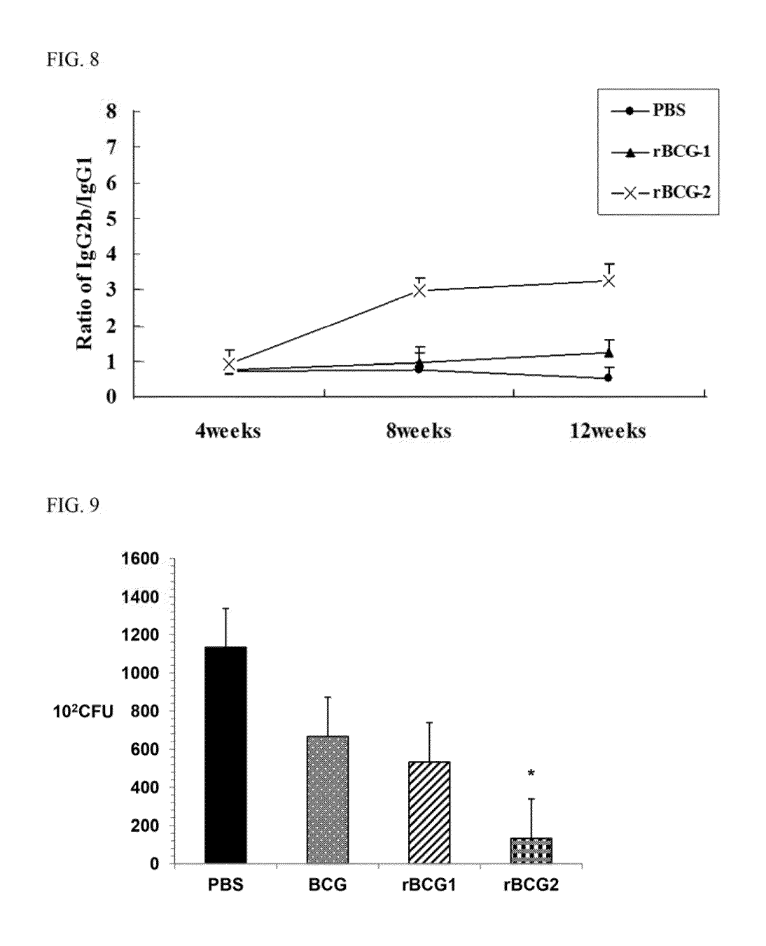

Groups of mice were immunized with the two different rBCG strains (rBCG::Ag85B-CFP10 and rBCG::Ag8SB-CFP10-IL12) and the control group was immunized with BCG. The level of antibody response in the sera of mice from different groups, using the recombinant purified Ag85B (FIG. 8) or CFP10 protein (data not shown) as the antigen. Compared with the BCG group, mice vaccinated with either of the two different rBCG strains induced higher levels of antibodies against Ag85B. In C57BL/6 mice, the gene coding for IgG2a is deleted; therefore, in the absence of a functional IgG2a gene, the IgG2b isotype was used as an indicator of the helper type 1 (Th1) response. The IgG2b/IgG1 ratios were calculated to determine the induction of Th1/Th2 responses in the animals. In contrast to the results for rBCG and BCG, the IgG2b/IgG1 ratios of mice immunized with rBCG-Ag85B-CFP10-IL12 were higher against the protein Ag85B at 8 and 12 weeks. Overall, the results revealed that the capability for induction of Th1/Th2 responses increased in the following sequence: BCG, rBCG::Ag85B-CFP10, and rBCG::Ag85B-CFP10-IL12.

### Evaluation Vaccine Protection in Mycobacterium Growth Inhibition Assay

It has been shown that the ability of splenocytes from vaccinated mice to inhibit bacterial intracellular growth is associated with vaccine protection. The test whether the increased immunogenicity shown by BCG or rBCG translated into greater inhibition of intracellular bacterial growth, we infected BMMφ with M. tuberculosis and, after washing for 4 h, added splenocyte cells from the spleens of mice that had been immunized 28 days previously with the different BCG strains. As a control, we observed that the addition of splenocytes from PBS treated animals provided no measurable benefit in controlling intracellular bacterial growth, as compared to no cells being added (FIG. 9). In contrast, T-cells from vaccinated animals contributed to control of M. tuberculosis growth. T-cell mediated killing of infected macrophages was more pronounced when T-cells from rBCG::Ag85B-CFP10-IL12, rBCG::Ag858-CFP10 (p≦0.01 for both) vaccinated mice were used as compared to T-cells from mice vaccinated with BCG.

rBCG Immunization Elicits IFN-γ Spot Forming Cells in Spleen and Lung

The immune responses were evaluated by characterization of peptide-specific CD4⁺ and CD8⁺ T cell response in lung and spleen, and showed that rBCG vaccines enhanced Th1 immunity compared with traditional BCG vaccine. In the present study, the protective ability of rBCG vaccines against M. tuberculosis has been conducted by determination of the ability of BCG or rBCG strains to elicit. IFN-γ releasing cells after immunization.

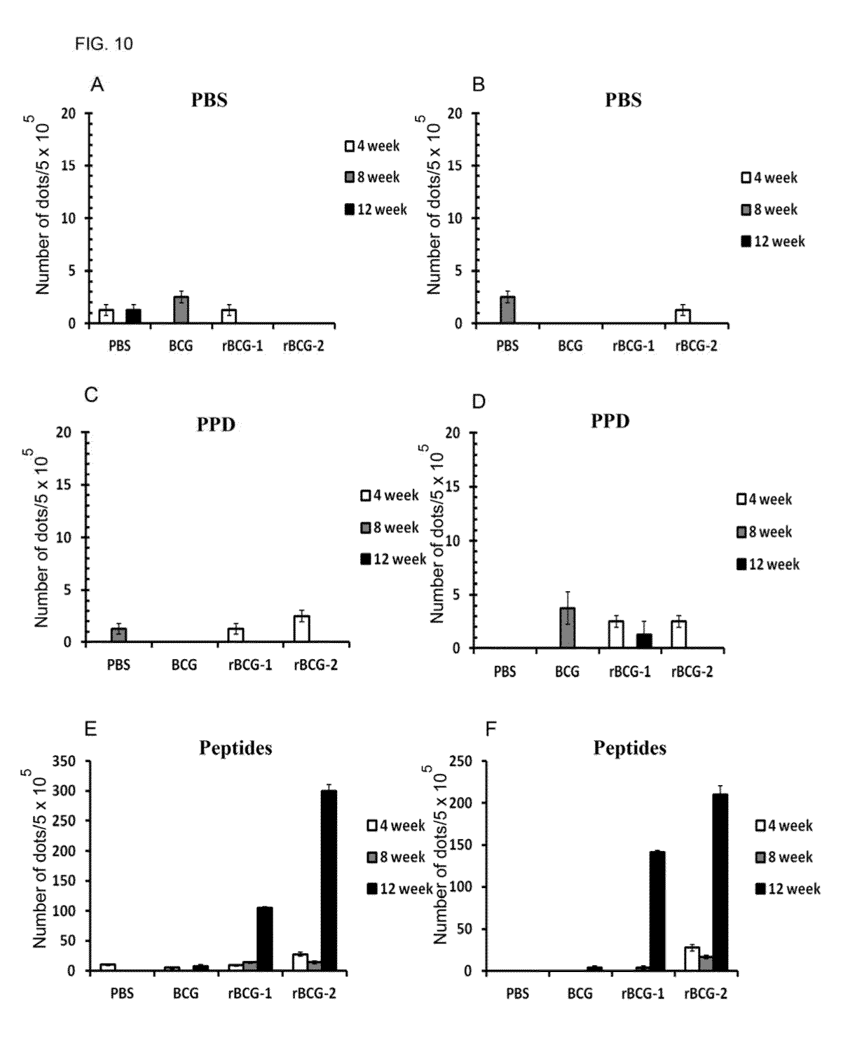

An ELISPOT assay was used to examine the relative numbers of IFN-γ-expressing cells in single cell suspension obtained from mice immunized with BCG or rBCG strains. The antigen, PPD or Ag85b/CFP10, were used to determine the cellular immunity after being vaccinated with BCG or rBCGs. FIG. 10 shows the results 4, 8 and 12 weeks after immunization with vaccine candidates. The results indicated that mouse spleen and lung cells immunized with rBCG1 or rBCG2 released IFN-γ more frequency compared with PBS control or BCG Tokyo 172 strain in response to Ag85b/CFP10 peptides (FIGS. 10E-F). Moreover, the number of IFN-γ expressing cells in the group of rBCG1 or rBCG2 reached the highest peak at 12 weeks (FIGS. 10E-F). No significant difference in all the groups stimulated by PBS or PPD was found (FIGS. 10A-D).

rBCG Immunization Promoted Th1 Type Immune Response

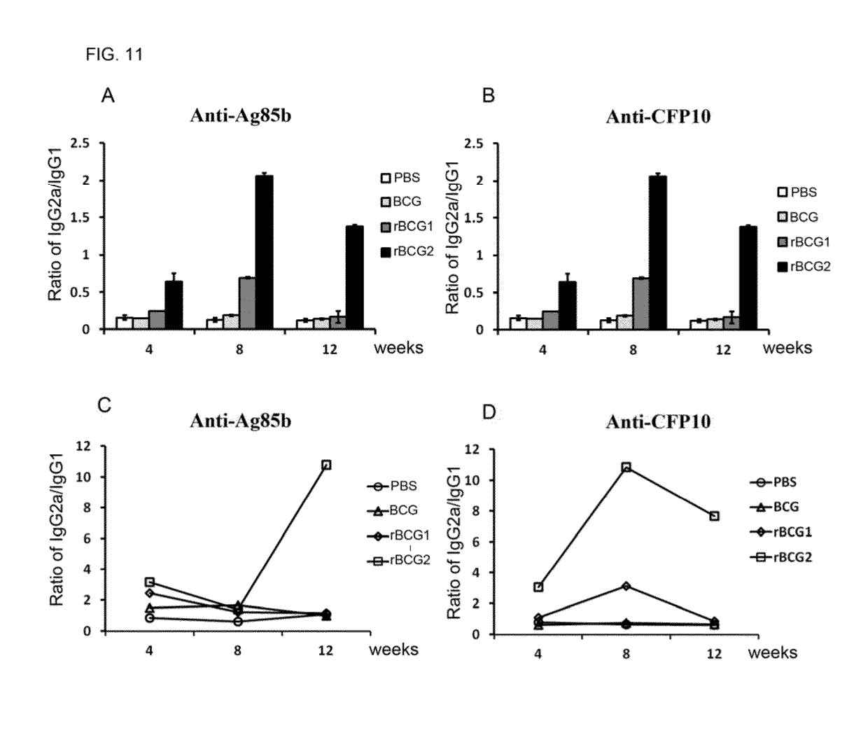

FIG. 11 illustrates the expressing level of antibody response in the sera of mice immunized with BCG, rBCG and control group-PBS, using the recombinant purified Ag85b or CFP10 protein as antigen. The mice immunized with this rBCGs strain group have resulted in generation of higher level of IgG2a antibody against Ag85b or CFP10 compared to the BCG strain group (FIGS. 11A-B). The highest titer of IgG2a antibody against Ag85b or CFP10 reached at 8 weeks, and declined at 12 weeks (FIGS. 11A-B).

The Th1 polarized arm of the cellular immune response is considered protective immunity against mycobacterium infection. The ratio of IgG2a/IgG1 was calculated to gauge the Th1/Th2 nature of the immune response in animals. In contrast to PBS control group or rBCG L the ratio was higher in the mice group immunized with rBCG2 whatever against the protein Ag85b or CFP10 (FIGS. 11C-D). The ratio of IgG2a/IgG1 in rBCG2 group against CFP10 at 8 weeks reached the highest peak that was similar to the trend of IgG2a antibody response (FIGS. 11B and D). However, the ratio in rBCG2 group against Ag85b was reaching the highest peak at 12 weeks (FIG. 11C). These results revealed that ability of the induction of Th1/Th2 immunity increased in the following list: PBS, BCG, rBCG1, rBCG2. Overall, a Th1-polarized response was initiated by rBCG vaccine.

rBCG Vaccination Limit M. tuberculosis H37Rv Multiplication in Macrophages

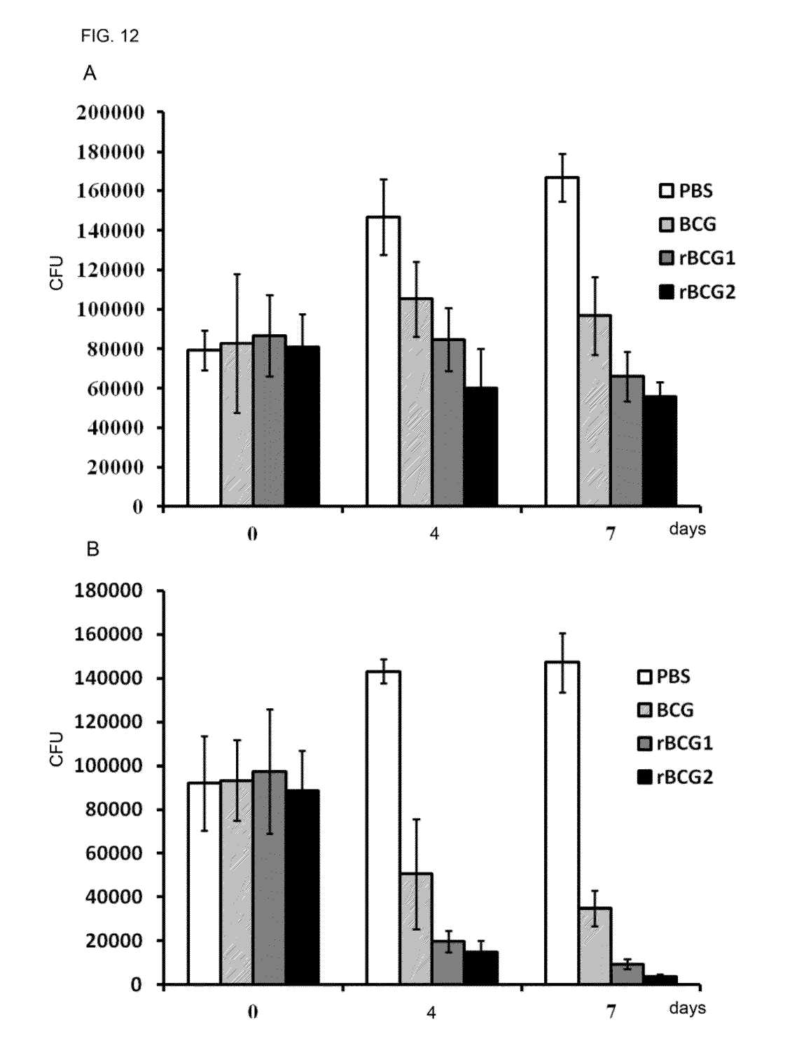

To evaluate the protective efficacy of the rBCG vaccines, the ability of the splenocytes isolated from vaccinated mice in inhibiting intracellular Mycobacterium growth in macrophages was performed. The splenocytes from mice immunized with rBCG1 and rBCG 2 significantly reduced the number of M. tuberculosis growth in macrophages when compared to the splenocytes from mice treated with PBS or BCG (FIGS. 12A and B). FIG. 12B revealed that mycobacterium growth in the co-culture having the splenocytes of the rBCG2 immunized animal group was reduced to about 30% and 10% on day 4 and 7 culture compared with that in the co-culture of the splenocyts of BCG-immunized animal group, indicating that rBCG2 vaccination elicited strong protective efficacy against M. tuberculosis infection (FIG. 12B).

Although the basic mechanisms of Immunity to M. tuberculosis are known, the details remain elusive. Animal models have demonstrated that T cells, rather than antibodies, are critical; in particular, CD4 T cells of the Th1 type, which are potent IFN-γ producers, are crucial for protection. There is increasing evidence from animal studies that CD8 T cells play a role in the control of TB, particularly as the infection progresses. Models of chronic infection suggest that these CD8 T cells are important in human disease, where the lag between infection and disease is frequently measured in years.

Infections lead to activation of innate immunity, followed by induction of the Th1 T-cell subset, which is thought to be induced in an antigen-specific fashion under the influence of IL-12. IL-12 can augment both innate and cellular immunity in many ways, which is potentially advantageous for the treatment of M. tuberculosis infection.

In all TB vaccine-related studies. BCG has been used as a gold standard to rate the effectiveness of a new vaccine candidate, even though it is the failure of BCG in the adult human population that has necessitated the development of a new TB vaccine in the first place. Thus, this convention suffers from a caveat—a new vaccine cannot progress to human trials without proving its superiority to BCG in animal models in which BCG works rather effectively. Consequently, it has been difficult to develop vaccines that give superior protection versus BCG in animal models.

Our vaccine simultaneously expresses IL-12 and two M. tuberculosis proteins, Ag85B and CFP-10, both of which have been individually validated previously to induce protective immune responses. Both antigens are being evaluated in clinical trials currently or are ready to enter trials shortly as single-agent entities or in combination. Our design strategy robustly expressed a spectrum of protective antigens simultaneously to induce a broad immune response that that can deliver a—multi-hit attack.

It is very encouraging that the Ag85B-CFP 10 plus IL-12 recombinant BCG vaccine promoted and sustained high levels of exactly these types of cytokine-coexpressing subsets and effectively protected against TB infection. Our results showed that the better immunogenicity and mycobacterial growth inhibition exhibited by rBCG::Ag85B-CFP 10 plus IL-12 than BCG may make the rBCG a preferred, vaccine candidate against TB.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A recombinant bacterial cell strain comprising:
(a) a first vector comprising a fusion transgene encoding Ag85B-CFP10 fusion protein, the fusion transgene being operably linked to a promoter effective for expression of the Ag85B-CFP10 fusion protein; and
(b) a second vector comprising a transgene encoding interleukin-12 (IL-12), the transgene being operably linked to a promoter effective for expression of the IL-12 protein.

2. The recombinant bacterial cell of claim 1, which expresses or overexpresses the Ag85B-CFP10 fusion protein and the IL-12 protein.

3. The recombinant, bacterial cell of claim 1, which is a recombinant Mycobacterium cell.

4. The recombinant bacterial cell of claim 3, which is a recombinant Mycobacterium bovis cell.

5. The recombinant bacterial cell of claim 3, which is a recombinant Bacille Calmette-Guerin (BCG).

6. The recombinant bacterial cell of claim 3, which is a recombinant Mycobacterium tuberculosis cell.

7. The recombinant bacterial cell of claim 1, wherein the fusion transgene is under the control of a heal shock protein (HSP) promoter.

8. The recombinant bacterial cell of claim 7, wherein the HSP promoter is a Mycobacterium bovis HSP60 promoter.

9. A recombinant Bacille Calmette-Guerin (BCG) vaccine strain, comprising:
(a) a fission transgene encoding Ag85B-CFP10 fusion protein, the fusion transgene being operably linked to a promoter effective for an increased expression of Ag85B and CFP10 proteins; and
(b) a transgene encoding IL-12 protein, the transgene being operably linked to a promoter effective for expression of the IL-12 protein.

10. The recombinant BCG vaccine strain of claim 9, wherein the fusion transgene is under the control of a heat shock protein (HSP) promoter.

11. The recombinant BCG vaccine strain of claim 10, wherein the HSP promoter is M. bovis HSP60 promoter.

12. The recombinant BCG vaccine strain of claim 9, which expresses the IL-12 protein in a non-fusion protein form.

13. The recombinant BCG vaccine strain of claim 9, which has at least one of the following characteristics:
(a) eliciting interferon γ-producing T cells and IgG2a antibody against Ag85b or CFP10; and
(b) conferring stronger protective efficacy against M. tuberculosis H37Rv Infection compared with BCG vaccine.

14. The recombinant BCG vaccine strain of claim 9, which expresses human IL-12 protein.

15. A method of inhibiting intracellular growth of Mycobacterium in a subject, comprising: administering to the subject the recombinant bacterial cell strain of claim 1 in an amount effective for inhibition of Mycobacterium growth in the lung cells of the subject.

16. The method of claim 15, wherein the subject has Mycobacterium tuberculosis infection.

17. A method of inhibiting intracellular growth of Mycobacterium in a subject, comprising: administering to the subject the recombinant BCG vaccine strain of claim 9 in an amount effective for causing an inhibition of Mycobacterium growth in the subject.

18. The method of claim 17, wherein the subject has Mycobacterium tuberculosis infection by M. tuberculosis H37Rv.

19. The method of claim 15, wherein the recombinant BCG vaccine strain causes an increased expression of interferon-γ and/or an increased number of CD4 and CD8 T cells in the spleen and/or lung tissue of the subject.

20. A recombinant Bacille Calmette-Guerin (BCG) vaccine strain, which expresses and secretes human IL-12 protein, and exhibits an increased expression of Ag85B and CFP10 proteins as compared to a BCG vaccine strain.
